(19) United States
(12) Patent Application Publication
(10) Pub. No.: US 2024/0183800 A1
(43) Pub. Date: Jun. 6, 2024

(54) Single Data Set Calibration and Imaging with Uncooperative Electromagnetic Inversion

(71) Applicants: GSI Electronique Inc, St-Hubert (CA); University of Manitoba, Winnipeg (CA)

(72) Inventors: Mohammad Asefi, Winnipeg (CA); Ian Jeffrey, Winnipeg (CA); Joe LoVetri, Winnipeg (CA); Colin Gerald Gilmore, Winnipeg (CA); Eungjoo Kim, Winnipeg (CA)

(21) Appl. No.: 18/551,719

(22) PCT Filed: Mar. 16, 2022
(86) PCT No.: PCT/IB2022/052392

§ 371 Date: Sep. 21, 2023

Related U.S. Application Data
(60) Provisional application No. 63/163,958, filed on Mar. 22, 2021.

(51) Int. Cl. G01N 22/04; G01N 33/02

(52) U.S. Cl. CPC G01N 22/04; G01N 33/02

(57) ABSTRACT

In one embodiment, a method, comprising: receiving measurement data of a container with contents stored within the container; performing a phaseless parametric inversion on the measurement data to provide a background model; determining calibration coefficients for each of a plurality of channels based on the measurement data and the background model; and determining calibrated scattered field measurements based on the background model and the calibration coefficients.

### CROSS REFERENCE TO RELATED APPLICATION

This application claims the benefit of U.S. Provisional Application No. 63/163,958, filed Mar. 22, 2021, which is hereby incorporated by reference in its entirety.

### TECHNICAL FIELD

The present disclosure is generally related to electromagnetic imaging of containers and, more particularly, a calibration system and method for electromagnetic inverse imaging systems.

### BACKGROUND

Imaging contents within a container is a powerful tool, especially when the interior of the container is difficult to access. Electromagnetic inverse imaging (EMI) technology estimates permittivity of a target and generates permittivity image maps of the target using electromagnetic signals. This EMI technology has been adapted to monitor grain storage bins. When storing grains, moisture of the grains should be controlled because grains that contain high moisture are likely to spoil. The electric permittivity is closely related to moisture. In general, the imaging system obtains the permittivity of grains by an EMI algorithm, and from that, the system may determine the moisture content in the grains and prompt and/or implement the appropriate action (e.g., if the system detects high moisture in the bin, the system activates a fan to reduce humidity).

One shortcoming to applying EMI technology to grain bin monitoring is the need for calibration of raw data. Signals that are received and transmitted are transferred between antennas and the main system via a Vector Network Analyzer (VNA), a switch, and cables. Inversion methods use computational models that generally do not take the measurement system into account and assume field measurements at a point, rather than the S-parameter (voltage ratios) measured by the VNAs. To be practically useful, grain bin imaging systems should produce meaningful target reconstructions when the state of the grain in the bin is unknown. However, unlike lab-based systems, calibrating grain bins with known targets is not practical, nor can the system be re-calibrated once filled with grain. Such imaging systems are sometimes referred to as uncooperative.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a schematic diagram that illustrates an example environment in which an embodiment of an example one-shot calibration system may be implemented.

FIG. 2 is a logical flow diagram that illustrates an embodiment of an example one-shot calibration method.

FIG. 3 is a block diagram that illustrates an embodiment of an example computing device of an example one-shot calibration system.

FIG. 4 is a flow diagram that illustrates an embodiment of an example one-shot calibration method.

### DESCRIPTION OF EXAMPLE EMBODIMENTS

### Overview

In one embodiment, a method, comprising: receiving measurement data of a container with contents stored within the container; performing a phaseless parametric inversion on the measurement data to provide a background model; determining calibration coefficients for each of a plurality of channels based on the measurement data and the background model; and determining calibrated scattered field measurements based on the background model and the calibration coefficients.

### Detailed Description

Certain embodiments of a one-shot calibration system and method are disclosed that provide for a single data set calibration for an uncooperative electromagnetic inverse imaging (EMI) system. The term, uncooperative, connotes a situation in which parameters or state of contents (e.g., specifications, including height of the contents along a container wall, cone angle, moisture content, temperature, etc.) are unknown. In certain embodiments of a one-shot calibration method, calibration is achieved via a single set of measurements, which in turn is used to ultimately perform a full three-dimensional (3D) inversion on the same set of measurements.

Digressing briefly, one challenge for industrial deployment of grain bin EMI systems is the calibration of raw data: electromagnetic measurements are made with a Vector Network Analyzer (VNA), signals are passed through a switch, down long cables, and finally reach the antennas inside a bin. This means that measurements are distorted by the presence of the measurement system. Inversion methods use computational models that generally do not take the measurement system into account and assume field measurements at a point, rather than the S-parameter (voltage ratios) measured by VNAs. To be practically useful, grain bin imaging systems should produce meaningful target reconstructions when the state of the grain in the bin is unknown (uncooperative). But as expressed above, field systems are unlike lab-based systems.

Further to the subject of calibration, typical EMI images are made from differential signals. S-parameter measurements, S_{xy}^{unknown}, for all pairs of transmitters x and receivers y are collected at time t₁ when there is a known state inside the imaging chamber (e.g., the grain is homogeneous and the grain surface can be inferred). A second measurement, S_{xy}^{unknown}, is taken at time t₂ when the grain has changed (e.g., moisture has started to increase). Standard calibration requires running a forward electromagnetic model on the known state, thus producing field estimates,H_{xy}^{known} (e.g., magnetic fields in amperes/meter, such as tangential surface magnetic fields). This known state is also referred to as a prior background model, and usually consists of the grain height, cone angle, and bulk permittivity. Using the known model, calibrated scattered field data, H_{xy}^{actual}(used as input to an inversion algorithm), are calculated as follows:

H_{xy}^{actual}=[(H_{xy}^{known})/(S_{xy}^{known})] (S_{xy}^{known}−S_{xy}^{unknown}).

This two-scan calibration procedure provides a way to compensate for the differences between the simulated model and the actual system, including the S-parameter-to-field conversion, the measurement system error and some differences between the real-world and the computational model. However, shortcoming to this prior approach includes the need for two measurements at different times. In other words, only changes in grain over time can be imaged. In contrast, certain embodiments of a one-shot calibration system calibrates an image with a single set of measurements, S_{xy}^{unknown}(e.g., without S_{xy}^{known}), negating the computational time in determining the calibrated scattered field data and hence the time and computational resources needed to achieve 3D full inversion imaging.

Having summarized certain features and/or benefits of a one-shot calibration system of the present disclosure, reference will now be made in detail to the description of a one-shot calibration system as illustrated in the drawings. While a one-shot calibration system will be described in connection with these drawings, there is no intent to limit it to the embodiment or embodiments disclosed herein. For instance, in the description that follows, one focus is on grain bin monitoring. However, certain embodiments of a one-shot calibration system may be used to determine other contents of a container, including one or any combination of other materials or solids, fluids, or gases, as long as such contents reflect electromagnetic waves. Further, although the description identifies or describes specifics of one or more embodiments, such specifics are not necessarily part of every embodiment, nor are all various stated advantages necessarily associated with a single embodiment or all embodiments. On the contrary, the intent is to cover all alternatives, modifications and equivalents included within the spirit and scope of the disclosure as defined by the appended claims. Further, it should be appreciated in the context of the present disclosure that the claims are not necessarily limited to the particular embodiments set out in the description.

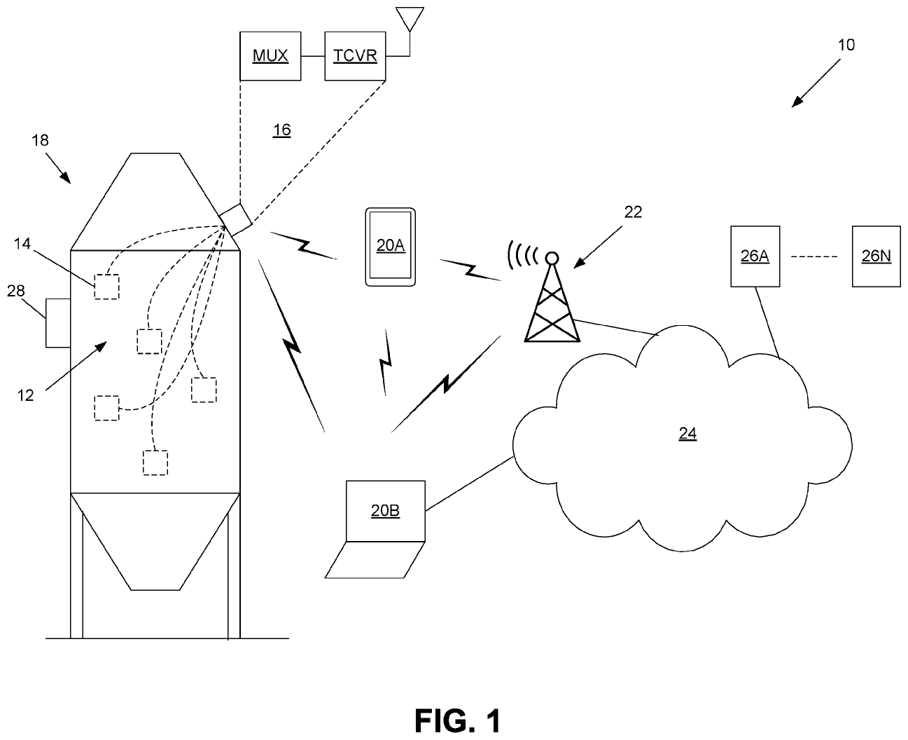

FIG. 1 is a schematic diagram that illustrates an example environment 10 in which an embodiment of a one-shot calibration system may be implemented. It should be appreciated by one having ordinary skill in the art in the context of the present disclosure that the environment 10 is one example among many, and that some embodiments of a one-shot calibration system may be used in environments with fewer, greater, and/or different components than those depicted in FIG. 1. The environment 10 comprises a plurality of devices that enable communication of information throughout one or more networks. The depicted environment 10 comprises an antenna array 12 comprising a plurality of antenna probes 14 and an antenna acquisition system 16 that is used to monitor contents within a container 18 and uplink with other devices to communicate and/or receive information. The container 18 is depicted as one type of grain storage bin (or simply, grain bin), though it should be appreciated that containers of other geometries, for the same (e.g., grain) or other contents, with a different arrangement (side ports, etc.) and/or quantity of inlet and outlet ports, may be used in some embodiments. As is known, electromagnetic imaging uses active transmitters and receivers of electromagnetic radiation to obtain quantitative and qualitative images of the complex dielectric profile of an object of interest (e.g., here, the contents or grain).

As shown in FIG. 1, multiple antenna probes 14 of the antenna array 12 are mounted along the interior of the container 18 in a manner that surrounds the contents to effectively collect the scattered signal. For instance, each transmitting antenna probe is polarized to excite/collect the signals scattered by the contents. That is, each antenna probe 14 illuminates the contents while the receiving antenna probes collect the signals scattered by the contents. The antenna probes 14 are connected (via cabling, such as coaxial cabling) to a radio frequency (RF) switch matrix or RF multiplexor (MUX) of the antenna acquisition system 16, the switch/mux switching between the transmitter/receiver pairs. That is, the RF switch/mux enables each antenna probe 14 to either deliver RF energy to the container 18 or collect the RF energy from the other antenna probes 14. The switch/mux is followed by an electromagnetic transceiver (TCVR) system of the antenna acquisition system 16 (e.g., a vector network analyzer or VNA). The electromagnetic transceiver system generates the RF wave for illumination of the contents of the container 18 as well as receives the measured fields by the antenna probes 14 of the antenna array 12. As the arrangement and operations of the antenna array 12 and antenna acquisition system 16 are known, further description is omitted here for brevity. Additional information may be found in the publications “Industrial scale electromagnetic grain bin monitoring”, Computers and Electronics in Agriculture, 136, 210-220, Gilmore, C., Asefi, M., Paliwal, J., & LoVetri, J., (2017), “Surface-current measurements as data for electromagnetic imaging within metallic enclosures”, IEEE Transactions on Microwave Theory and Techniques, 64, 4039, Asefi, M., Faucher, G., & LoVetri, J. (2016), and “A 3-d dual-polarized near-field microwave imaging system”, IEEE Trans. Microw. Theory Tech., Asefi, M., OstadRahimi, M., Zakaria, A., LoVetri, J. (2014).

Note that in some embodiments, the antenna acquisition system 16 may include additional circuitry, including a global navigation satellite systems (GNSS) device or triangulation-based devices, which may be used to provide location information to another device or devices within the environment 10 that remotely monitors the container 18 and associated data. The antenna acquisition system 16 may include suitable communication functionality to communicate with other devices of the environment.

The uncalibrated, raw data collected from the antenna acquisition system 16 is communicated (e.g., via uplink functionality of the antenna acquisition system 16) to one or more electronic devices of the environment 10, including electronic devices 20A and/or 20B. Communication by the antenna acquisition system 16 may be achieved using near field communications (NFC) functionality, Blue-tooth functionality, 802.11-based technology, satellite technology, streaming technology, including LoRa, and/or broadband technology including 3G, 4G, 5G, etc., and/or via wired communications (e.g., hybrid-fiber coaxial, optical fiber, copper, Ethernet, etc.) using TCP/IP, UDP, HTTP, DSL, among others. The electronic devices 20A and 20B communicate with each other and/or with other devices of the environment 10 via a wireless/cellular network 22 and/or wide area network (WAN) 24, including the Internet. The wide area network 24 may include additional networks, including an Internet of Things (IOT) network, among others. Connected to the wide area network 24 is a computing system comprising one or more servers 26 (e.g., 26A, . . . 26N).

The electronic devices 20 may be embodied as a smartphone, mobile phone, cellular phone, pager, stand-alone image capture device (e.g., camera), laptop, tablet, personal computer, workstation, among other handheld, portable, or other computing/communication devices, including communication devices having wireless communication capability, including telephony functionality. In the depicted embodiment of FIG. 1, the electronic device 20A is illustrated as a smartphone and the electronic device 20B is illustrated as a laptop for convenience in illustration and description, though it should be appreciated that the electronic devices 20 may take the form of other types of devices as explained above.

The electronic devices 20 provide (e.g., relay) the (uncalibrated, raw) data sent by the antenna acquisition system 16 to one or more servers 26 via one or more networks. The wireless/cellular network 22 may include the necessary infrastructure to enable wireless and/or cellular communications between the electronics device 20 and the one or more servers 26. There are a number of different digital cellular technologies suitable for use in the wireless/cellular network 22, including: 3G, 4G, 5G, GSM, GPRS, CDMAOne, CDMA2000, Evolution-Data Optimized (EV-DO), EDGE, Universal Mobile Telecommunications System (UMTS), Digital Enhanced Cordless Telecommunications (DECT), Digital AMPS (IS-136/TDMA), and Integrated Digital Enhanced Network (iDEN), among others, as well as Wireless-Fidelity (Wi-Fi), 802.11, streaming, etc., for some example wireless technologies.

The wide area network 24 may comprise one or a plurality of networks that in whole or in part comprise the Internet. The electronic devices 20 may access the one or more server 26 via the wireless/cellular network 22, as explained above, and/or the Internet 24, which may be further enabled through access to one or more networks including PSTN (Public Switched Telephone Networks), POTS, Integrated Services Digital Network (ISDN), Ethernet, Fiber, DSL/ADSL, Wi-Fi, among others. For wireless implementations, the wireless/cellular network 22 may use wireless fidelity (Wi-Fi) to receive data converted by the electronic devices 20 to a radio format and process (e.g., format) for communication over the Internet 24. The wireless/cellular network 22 may comprise suitable equipment that includes a modem, router, switching circuits, etc.

The servers 26 are coupled to the wide area network 24, and in one embodiment may comprise one or more computing devices networked together, including an application server(s) and data storage. In one embodiment, the servers 26 may serve as a cloud computing environment (or other server network) configured to perform processing required to implement an embodiment of a one-shot calibration system as well as pixel-based inversion. When embodied as a cloud service or services, the server 26 may comprise an internal cloud, an external cloud, a private cloud, a public cloud (e.g., commercial cloud), or a hybrid cloud, which includes both on-premises and public cloud resources. For instance, a private cloud may be implemented using a variety of cloud systems including, for example, Eucalyptus Systems, VMWare vSphere®, or Microsoft® HyperV. A public cloud may include, for example, Amazon EC2®, Amazon Web Services®, Terremark®, Savvis®, or GoGrid®. Cloud-computing resources provided by these clouds may include, for example, storage resources (e.g., Storage Area Network (SAN), Network File System (NFS), and Amazon S3®), network resources (e.g., firewall, load-balancer, and proxy server), internal private resources, external private resources, secure public resources, infrastructure-as-a-services (laaSs), platform-as-a-services (PaaSs), or software-as-a-services (SaaSs). The cloud architecture of the servers 26 may be embodied according to one of a plurality of different configurations. For instance, if configured according to MICROSOFT AZURE™, roles are provided, which are discrete scalable components built with managed code. Worker roles are for generalized development, and may perform background processing for a web role. Web roles provide a web server and listen for and respond to web requests via an HTTP (hypertext transfer protocol) or HTTPS (HTTP secure) endpoint. VM roles are instantiated according to tenant defined configurations (e.g., resources, guest operating system). Operating system and VM updates are managed by the cloud. A web role and a worker role run in a VM role, which is a virtual machine under the control of the tenant. Storage and SQL services are available to be used by the roles. As with other clouds, the hardware and software environment or platform, including scaling, load balancing, etc., are handled by the cloud.

In some embodiments, the servers 26 may be configured into multiple, logically-grouped servers (run on server devices), referred to as a server farm. The servers 26 may be geographically dispersed, administered as a single entity, or distributed among a plurality of server farms. The servers 26 within each farm may be heterogeneous. One or more of the servers 26 may operate according to one type of operating system platform (e.g., WINDOWS-based O.S., manufactured by Microsoft Corp. of Redmond, Wash.), while one or more of the other servers 26 may operate according to another type of operating system platform (e.g., UNIX or Linux). The group of servers 26 may be logically grouped as a farm that may be interconnected using a wide-area network connection or medium-area network (MAN) connection. The servers 26 may each be referred to as, and operate according to, a file server device, application server device, web server device, proxy server device, or gateway server device.

In one embodiment, one or more of the servers 26 may comprise a web server that provides a web site that can be used by users interested in the contents of the container 18 via browser software residing on an electronic device (e.g., electronic device 20). For instance, the web site may provide visualizations that reveal permittivity of the contents and/or geometric and/or other information about the container and/or contents (e.g., the volume geometry, such as cone angle, height of the grain along the container wall, etc.).

The functions of the servers 26 described above are for illustrative purpose only. The present disclosure is not intended to be limiting. For instance, functionality for performing the one-shot calibration method and/or pixel-based inversion may be implemented at a computing device that is local to the container 18 (e.g., edge computing), or in some embodiments, such functionality may be implemented at the electronic device(s) 20. In some embodiments, functionality of the one-shot calibration method and/or pixel-based inversion may be implemented in different devices of the environment 10 operating according to a primary-secondary configuration or peer-to-peer configuration. In some embodiments, the antenna acquisition system 16 may bypass the electronic devices 20 and communicate with the servers 26 via the wireless/cellular network 22 and/or the wide area network 24 using suitable processing and software residing in the antenna acquisition system 16.

Note that cooperation between the electronic devices 20 (or in some embodiments, the antenna acquisition system 16) and the one or more servers 26 may be facilitated (or enabled) through the use of one or more application programming interfaces (APIs) that may define one or more parameters that are passed between a calling application and other software code such as an operating system, a library routine, and/or a function that provides a service, that provides data, or that performs an operation or a computation. The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer employs to access functions supporting the API. In some implementations, an API call may report to an application the capabilities of a device running the application, including input capability, output capability, processing capability, power capability, and communications capability.

An embodiment of a one-shot calibration system may include any one or a combination of the components of the environment 10. For instance, in one embodiment, the one-shot calibration system may include a single computing device (e.g., one of the servers 26 or one of the electronic devices 20), and in some embodiments, the one-shot calibration system may comprise the antenna array 12, the antenna acquisition system 16, and one or more of the server 26 and/or electronic devices 20. For purposes of illustration and convenience, implementation of an embodiment of a one-shot calibration method is described in the following as being implemented in a computing device that may be one of the servers 26, with the understanding that functionality may be implemented in other and/or additional devices. Also shown in FIG. 1 is a moisture-affecting device 28 (e.g., a fan, blower, etc.) operably coupled (e.g., directly mounted, ducted, etc.) to the container 18 that may be activated by one of the devices (e.g., server 26, electronic device 20) based on a determination of the moisture content within the container 18 (e.g., if there is too much moisture in the grain). Though a single moisture-affecting device 28 is shown, there may be a plurality of such devices.

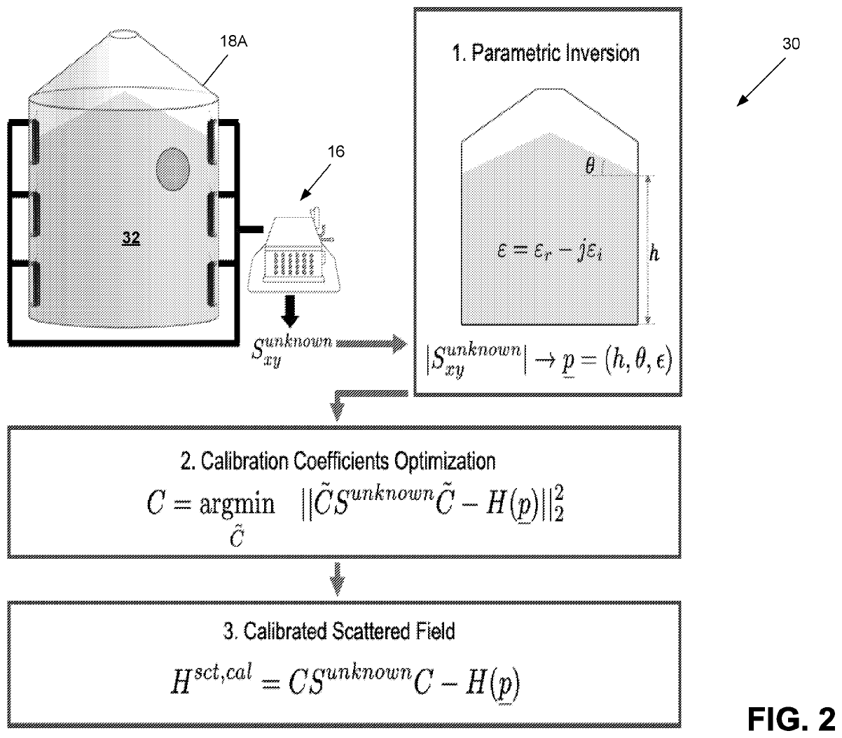

In one example operation, a user (via the electronic device 20) requests measurements of the contents of the container 18. This request is communicated to the antenna acquisition system 16. In some embodiments, the triggering of measurements may occur automatically based on a fixed time frame or based on certain conditions or based on detection of an authorized user (electronic) device 20. In some embodiments, the request may trigger the communication of measurements that have already occurred. The antenna acquisition system 16 activates (e.g., excites) the antenna probes 14 of the antenna array 12, such that the acquisition system (via the transmission of signals and receipt of the scattered signals) collects a set of raw, uncalibrated electromagnetic data at a set of (a plurality of) discrete, sequential frequencies (e.g., 10-100 Mega-Hertz (MHZ), though not limited to this range of frequencies nor limited to collecting the frequencies in sequence). In one embodiment, the uncalibrated data comprises total-field, S-parameter measurements (which are used to generate a background model or information as described below). As is known, S-parameters are ratios of voltage levels (e.g., due to the decay between the sending and receiving signal). Though S-parameter measurements are described, in some embodiments, other mechanisms for describing voltages on a line may be used. For instance, power may be measured directly (without the need for phase measurements), or various transforms may be used to convert S-parameter data into other parameters, including transmission parameters, impedance, admittance, etc. Since the uncalibrated S-parameter measurement is corrupted by the switching matrix and/or varying lengths and/or other differences (e.g., manufacturing differences) in the cables connecting the antenna probes 14 to the antenna acquisition system 16, it is important that certain embodiments of the one-shot calibration method use only magnitude (i.e., phaseless) data as input. Further, as expressed above, certain embodiments of the one-shot calibration method account for distortions from the measurement system, unlike prior approaches. The antenna acquisition system 16 communicates (e.g., via a wired and/or wireless communications medium) the uncalibrated (S-parameter) data to the electronic device 20, which in turn communicates the uncalibrated data to the server 26. At the server 26, data processing performed as described in association with FIG. 2.

Referring now to FIG. 2, shown is a logical flow diagram 30 that illustrates an embodiment of an example one-shot calibration method. Blocks 1-3 of the logical flow diagram 30 are intended to represent modules of code (e.g., opcode, machine language code, higher level code), fixed or programmable hardware, or a combination of both that implement the functionality or method step of each block, where all blocks may be implemented in a single component or device or implemented using a distributed network of components or devices. Continuing, the logical flow diagram 30 comprises the container 18A with contents 32 (e.g., grain) stored therein, the antenna acquisition system 16A (e.g., switch and vector analyzer), and blocks 1-3, which include parametric inversion (1), calibration coefficients optimization (2), and calibrated scattered field (3). The container 18A is depicted with six (6) antenna probes installed on the inside wall of the container 18A for ease of illustration, with the understanding by one skilled in the art that a different quantity of probes and/or different arrangement may be used. For instance, raw data acquired by the antenna probes are communicated through coaxial cables out of the container 18A to the switch and vector analyzer of the antenna acquisition system 16. Blocks 1-3 may be implemented in one or more devices, including a computational device of the antenna acquisition system 16, electronic device(s) 20, and/or the server 26.

In one embodiment of a one-shot calibration method illustrated in blocks 1-3, with only one set of measurements S_{xy}^{unknown} available, one initial step is obtaining a simple background model from which scattered fields may be generated. Once a background model has been determined, calibration for system/model effects (e.g., different cable lengths) can be implemented. More particularly, and referring to block 1, the one-shot calibration system performs a phaseless parametric inversion with the raw measurements to obtain the known background model. The known background model consists of the grain height at the bin wall h, cone angle ⊖, and bulk average complex-valued permittivity ε=εr−jεi. A method of obtaining the known background model is achieved via a phaseless parametric inversion on the parameters p=(h, ⊖, ε). To determine these parameters, raw S_{xy}^{unknown} measurements are taken and then the following cost functional is minimized according to Eqn. 1:

$$\underline{p} = \text{?} {‖ \vert {α}_{x} ( \underline{\tilde{p}} ) \text{?} \vert - \vert {H}_{xy} ( \underline{\tilde{p}} ) \vert ‖}_{2}^{2} , \qquad ( 1 )$$

$$\text{?} \text{indicates text missing or illegible when filed}$$

where αₓ is a per-transmitter factor used to scale average signal levels between forward-solver-generated estimate fields H_{xy}(p) and the VNA measurements S_{xy}^{unknown} given by Eqn. 2 below:

αₓ(p)=(Σ_{y}|H_{xy}(p)|)/(Σ_{y}|S_{xy}^{unknown}|). (2)

By using phaseless data and minimizing this objective function, parameters p are obtained, which provide a bulk estimate of the bin (container 18A) contents.

Referring to block 2, a next step in an embodiment of the one-shot calibration method comprises determining calibration coefficients. In other words, the one-shot calibration method calibrates the S_{xy}^{unknown} . The calibration uses a set of per-channel calibration coefficients. For instance, in the case of a grain bin with twenty-four (24) antennas, twenty-four (24) calibration coefficients cₓ are sought (similarly, for the example six (6) antenna probe configuration shown illustratively in FIG. 2, six (6) calibration coefficients are sought). Notation is simplified by representing these coefficients as a diagonal calibration matrix C (e.g., along the diagonal, c₁, c₂, . . . c_{N}), where N is the number of antennas or antenna probes (i.e. transmit/receive channels) and cₓ is the (complex) calibration coefficient for channel x used to capture channel loss and phase shift. The diagonal calibration matrix C is calculated according to Eqn. 3 below:

$$C = \text{?} {‖ \text{?} \tilde{C} - H ( \underline{p} ) ‖}_{2}^{2} , \qquad ( 3 )$$

$$\text{?} \text{indicates text missing or illegible when filed}$$

where S^{unknown} is the entire matrix of S_{xy}^{unknown} (H (p) is defined analogously). The quantity (\tilde{C}S^{unknown} \tilde{C})_{xy}=cₓS_{xy}^{unknown} c_{y} and the coefficients cₓ and c_{y} serve to account for cable loss and phase shifts along the channels x and y in the measurement path that are not accounted for in the forward model used to generate H (p). This per-channel calibration model is justified, since a significant portion of signal modification due to the measurement system is due to a magnitude and phase shift through each transmit/receive channel. Further, this channel phase shift and loss are the same whether the channel is in a transmit mode or receive mode. In one embodiment, coefficients are obtained using L2 norm minimization with raw measurements and the result from Step 1. In general, the inputs to the example minimization formula shown in FIG. 2 comprise the result of a bulk solve (e.g., which output grain height, cone angle, moisture content) and the measured data itself (e.g., complex field data or complex S-parameters). In some embodiments, other minimization techniques known to those having ordinary skill in the art may be used. Note that in some embodiments, cross-channel signal leakage (that occurs primarily inside the switch) may be ignored, since a switch may be used that is specifically designed (e.g., use of ground pins, reducing the signal to ground ratio, etc.) to minimize cross-channel signals. The calibration matrix effectively assumes that each transmit/receive channel can be viewed as a lossy transmission line (not a full two-port device between the VNA and the antenna). The diagonal C-matrix also takes into account the antenna factor (that compensates for the change between the field and voltage ratio measurements).

Referring to logical block diagram 3, an embodiment of the one-shot calibration method determines the calibrated scattered measurements. That is, once the per-channel calibration coefficients have been calculated, the calibrated scattered field measurements H_{xy}^{actual} are computed according to Eqn. 4 below:

H^{actual}=CS^{unknown}C−H(p). (4)

The calibrated scattered fields are summarized as the channel compensated difference between a single set of measurements S^{unknown} and a simple parametric model corresponding to those same measurements. Once the one-shot calibration method has been applied to produce H^{actual}, an inversion algorithm can be applied to detect hotspots (e.g., areas of high moisture content) in the stored grain. In one embodiment, a parallel 3D Finite-Element Contrast Source Inversion Method (FEM-CSI) may be used. Further information on CSI may be found in published literature, including “Full vectorial parallel finite-element contrast source inversion method” by A. Zakaria, I. Jeffrey, and J. Lovetri, published in 2013 in Prog. Electromagn. Res., vol. 142, pp. 463-384.

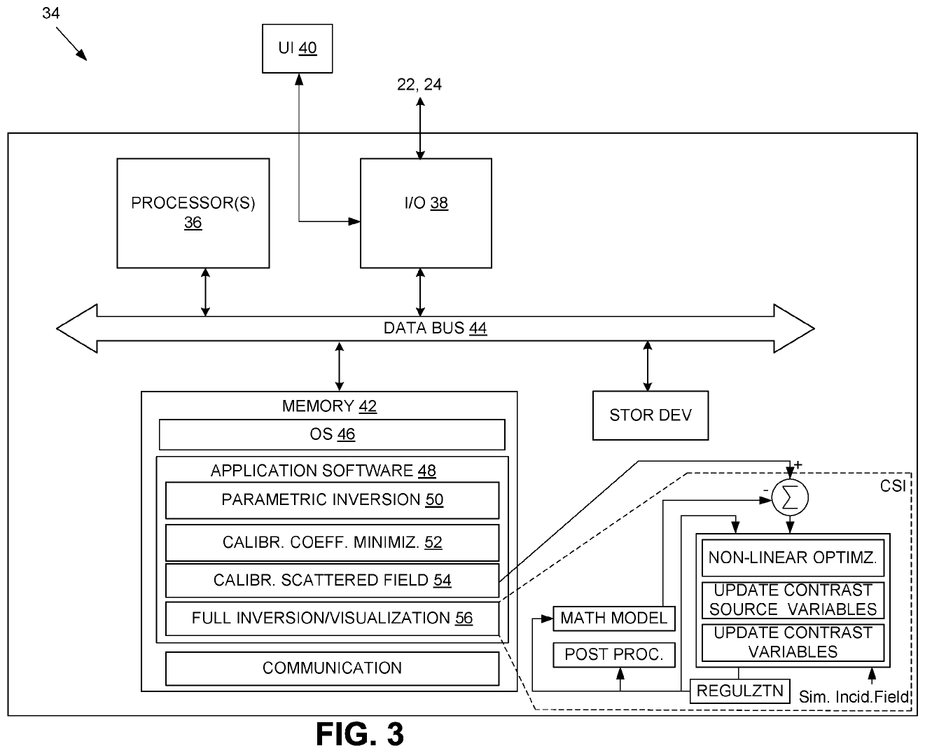

Having described an embodiment of a one-shot calibration method, attention is directed to FIG. 3, which illustrates an example computing device 34 that comprises an embodiment of an example one-shot calibration system. In one embodiment, the computing device 34 may provide functionality for one or more of the servers 26 and/or one of the electronic devices 20. Though described as implementing certain functionality of a one-shot calibration method, in some embodiments, such functionality may be distributed among plural devices (e.g., using plural, distributed processors) that are co-located or geographically dispersed. In some embodiments, functionality of the computing device 34 may be implemented in another device, including a programmable logic controller, application-specific integrated circuit (ASIC), field-programmable gate array (FPGA), among other processing devices. It should be appreciated that certain well-known components of computers are omitted here to avoid obfuscating relevant features of computing device 34. In one embodiment, the computing device 34 comprises one or more processors, such as processor 36, input/output (I/O) interface(s) 38, a user interface 40, and memory 42, all coupled to one or more data busses, such as data bus 44. The memory 42 may include any one or a combination of volatile memory elements (e.g., random-access memory RAM, such as DRAM, and SRAM, etc.) and nonvolatile memory elements (e.g., ROM, hard drive, tape, CDROM, etc.). The memory 42 may store a native operating system, one or more native applications, emulation systems, or emulated applications for any of a variety of operating systems and/or emulated hardware platforms, emulated operating systems, etc. In the embodiment depicted in FIG. 3, the memory 42 comprises an operating system 46 and application software 48.

In one embodiment, the application software 48 comprises parametric inversion module 50, calibration coefficient minimization/optimization module 52, calibrated scattered field module 54, and full inversion/visualization module 56. Functionality for modules 50, 52, and 54 are described above in association with FIG. 2, and hence further description of the same is omitted here for brevity. Memory 42 further comprises a communications module that formats data according to the appropriate format to enable transmission or receipt of communications over the networks and/or wireless or wired transmission hardware (e.g., radio hardware). In general, the application software 48 performs the functionality described in association with the logical flow diagram 30 (FIG. 2).

The full inversion/visualization module 56 may comprise known pixel-based inversion (PBI) software. For instance, the full inversion/visualization module 56 comprises known algorithms for performing pixel-based inversion based on the outputs provided by the calibrated scattered field module 54, and includes contrast source inversion or other known visualization software. For instance, FEM-CSI may be implemented, as schematically illustrated in FIG. 3. Digressing briefly, in general, the illuminated scattered field is measured at multiple receiver locations around an object of interest on measurement surface, the object of interest represented using complex-valued relative permittivity εr(r) as a function of position, which is converted to the so-called contrast function, reproduced below as Eqn. 5:

$$\text{?} ( r ) \text{?} \frac{ε r - ε rb}{ε rb} , \qquad ( 5 )$$

$$\text{?} \text{indicates text missing or illegible when filed}$$

which for an air background, εrb=1 simply becomes εr−1. A final goal in the full inversion process is to reconstruct the relative permittivity εr of an object of interest from measured data on measurement surface S, where generally, iterative methods are used to iterate between solving for the contrast using an assumed total-field and solving for the total field in a domain equation using an assumed contrast. In CSI, as is known, the measured scattered field data and a functional over the imaging domain are combined within an objective function that is minimized with respect to both unknowns. For instance, when the CSI cost functional is used, the CSI cost functional is formulated using the contrast sources, which vary with transmitter and the contrast, and which is constructed as the sum of normalized data-error and domain-error functionals. For each transmitter, one component of the cost function is the norm of the difference of the measured scattered field data and the calculated scattered field at the receiver locations. Assuming a finite-element forward model, computation of one functional component of the CSI cost functional involves a matrix (the inverse of an FEM matrix operator that transforms contrast source variables (w(r)= [symbol] (r)Eₜₒₜₐₗ (r)) of an imaging domain to scattered field values within a whole domain (problem domain)) and a matrix operator (transforms field values from the whole domain to receiver locations on the measurement surface S). The other functional component (sometimes referred to as a Maxwellian regularizer, formulated using the forward model) of the CSI cost functional is a functional over the imaging domain and is calculated using an FEM model of an incident field within the imaging domain as well as the contrast, [symbol] , and contrast sources w(r), where a matrix operator transforms field values from the problem domain to points inside the imaging domain. The CSI objective functional, F^{CSI}( [symbol] , w(r)) is minimized by updating the contrast sources and the contrast variables sequentially in an iterative fashion using a conjugate gradient technique. This process is generally and schematically illustrated in FIG. 3, though known to those having ordinary skill in the art as detailed further in the referenced publication cited above. That is, as CSI is well understood in the industry, further description of the same is omitted here for brevity. Visualization may include parameter values describing permittivity (and/or other content parameters) and geometric information about the contents, including the height of the grain along the container wall, the angle of grain repose, and the average complex permittivity of the grain. In some embodiments, the rendering of the color of the grain may be indicative of average grain moisture content, among other parameters.

In some embodiments, one or more functionality of the application software 48 may be implemented in hardware. In some embodiments, one or more of the functionality of the application software 48 may be performed in more than one device. It should be appreciated by one having ordinary skill in the art that in some embodiments, additional or fewer software modules (e.g., combined functionality) may be employed in the memory 42 or additional memory. In some embodiments, a separate storage device may be coupled to the data bus 44, such as a persistent memory (e.g., optical, magnetic, and/or semiconductor memory and associated drives).

The processor 36 may be embodied as a custom-made or commercially available processor, a central processing unit (CPU), graphic processing unit (GPU), or an auxiliary processor among several processors, a semiconductor based microprocessor (in the form of a microchip), a macroprocessor, one or more ASICs, a plurality of suitably configured digital logic gates, and/or other well-known electrical configurations comprising discrete elements both individually and in various combinations to coordinate the overall operation of the computing device 34.

The I/O interfaces 38 provide one or more interfaces to the networks 22 and/or 24. In other words, the I/O interfaces 38 may comprise any number of interfaces for the input and output of signals (e.g., analog or digital data) for conveyance over one or more communication mediums.

The user interface (UI) 40 may be a keyboard, mouse, microphone, touch-type display device, head-set, and/or other devices that enable visualization of the contents and/or container as described above. In some embodiments, the output may include other or additional forms, including audible or on the visual side, rendering via virtual reality or augmented reality based techniques.

Note that in some embodiments, the manner of connections among two or more components may be varied. Further, the computing device 34 may have additional software and/or hardware, or fewer software.

The application software 48 comprises executable code/instructions that, when executed by the processor 36, causes the processor 36 to implement the functionality shown and described in association with the one-shot calibration method, including functionality described in association with FIG. 2, and full inversion/visualization (in part via the user interface 40). As the functionality of the application software 48 has been described in the description corresponding to the aforementioned figures, further description here is omitted to avoid redundancy. In some embodiments, the application software 48 may be used to activate a moisture-affecting device (e.g., moisture-affecting device 28) based on the results of computations.

Execution of the application software 48 is implemented by the processor 36 under the management and/or control of the operating system 46. In some embodiments, the operating system 46 may be omitted. In some embodiments, functionality of application software 48 may be distributed among plural computing devices (and hence, plural processors).

When certain embodiments of the computing device 34 are implemented at least in part with software (including firmware), as depicted in FIG. 3, it should be noted that the software can be stored on a variety of non-transitory computer-readable medium (including memory 42) for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the computing device 34 are implemented at least in part with hardware, such functionality may be implemented with any or a combination of the following technologies, which are all well-known in the art: a discrete logic circuit(s) having logic gates for implementing logic functions upon data signals, an ASIC having appropriate combinational logic gates, a programmable gate array(s) (PGA), a field programmable gate array (FPGA), etc.

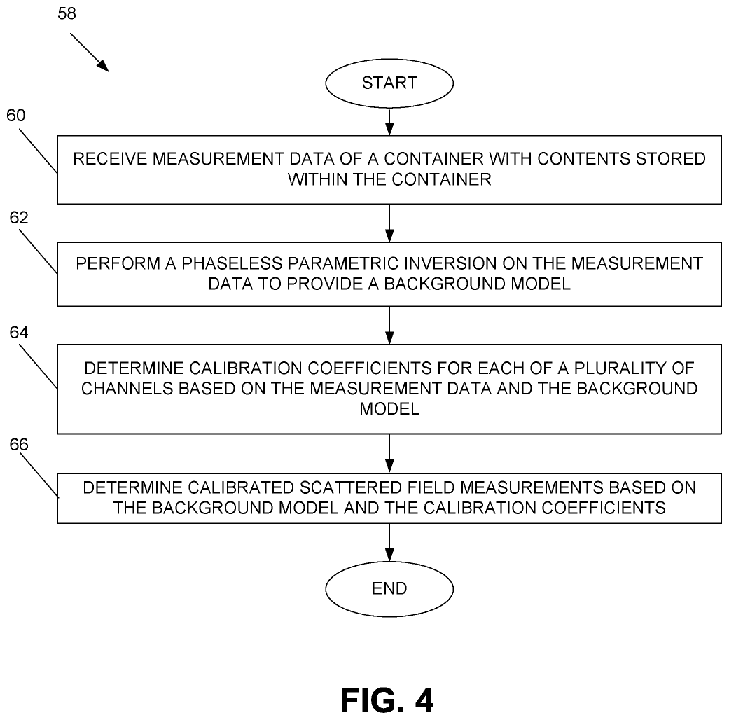

Having described certain embodiments of a one-shot calibration system and method, it should be appreciated within the context of the present disclosure that one embodiment of a one-shot calibration method, denoted as method 58 and illustrated in FIG. 4, and implemented using one or more processors (e.g., of a computing device or plural computing devices), comprises receiving measurement data of a container with contents stored within the container (60); performing a phaseless parametric inversion on the measurement data to provide a background model (62); determining calibration coefficients for each of a plurality of channels based on the measurement data and the background model (64); and determining calibrated scattered field measurements based on the background model and the calibration coefficients (66).

Any process descriptions or blocks in flow diagrams should be understood as representing logic (software and/or hardware) and/or steps in a process, and alternate implementations are included within the scope of the embodiments in which functions may be executed out of order from that shown or discussed, including substantially concurrently, or with additional steps (or fewer steps), depending on the functionality involved, as would be understood by those reasonably skilled in the art of the present disclosure.

Certain embodiments of a one-shot calibration system improves upon EMI systems that use two measurement data sets, the one-shot calibration system using only one set of S-parameters for both calibration and imaging. Further, certain embodiments of the one-shot calibration system provides an efficient method for imaging inhomogeneities with a single measurement in an uncooperative EMI system, including grain bins. As explained above, existing systems need a minimum of two data sets to achieve imaging, including one for a well-known state of the bin and the commodity in the bin for purposes of calibration (which is difficult and impractical to obtain in most instances), and one from any other day to obtain an image. With a single data set calibration, the calibration data is obtained from the data set intended to be imaged upon, thus eliminating the need for multiple data sets to obtain a single image and speeding up the imaging process.

It should be emphasized that the above-described embodiments of the present disclosure are merely possible examples of implementations, merely set forth for a clear understanding of the principles of the disclosure. Many variations and modifications may be made to the above-described embodiment(s) of the disclosure without departing substantially from the scope of the disclosure. All such modifications and variations are intended to be included herein within the scope of this disclosure and protected by the following claims.

1. A system, comprising:
one or more processors; and
a memory comprising instructions, wherein the one or more processors are configured by the instructions to:
receive measurement data of a container with contents stored within the container;
perform a phaseless parametric inversion on the measurement data to provide a background model;
determine calibration coefficients for each of a plurality of channels based on the measurement data and the background model; and
determine calibrated scattered field measurements based on the background model and the calibration coefficients.

2. The system of claim 1, wherein the measurement data comprises a set of S-measurements, wherein the same set of S-measurements is used for calibration and imaging of the contents.

3. The system of claim 1, wherein the background model comprises content height at a wall of the container, cone angle formed by the contents, and bulk average permittivity of the contents.

4. The system of claim 3, wherein the container comprises a grain storage bin, and the contents is grain.

5. The system of claim 1, wherein the calibration coefficients correspond to channel loss and phase shift.

6. The system of claim 1, wherein the one or more processors are configured by the instructions to determine calibration coefficients by performing a minimization with the measurement data and the background model.

7. The system of claim 6, wherein the minimization comprises an L2 norm minimization.

8. The system of claim 1, wherein the one or more processors are further configured by the instructions to perform full inversion based on the calibrated scattered field measurements and the measurement data and one or more of provide an image map of the contents or activate a moisture-affecting device based on the full inversion.

9. The system of claim 8, wherein the image map comprises a 3D permittivity map.

10. A method, comprising:
receiving measurement data of a container with contents stored within the container;
performing a phaseless parametric inversion on the measurement data to provide a background model;
determining calibration coefficients for each of a plurality of channels based on the measurement data and the background model; and
determining calibrated scattered field measurements based on the background model and the calibration coefficients.

11. The method of claim 10, wherein the measurement data comprises a set of S-measurements, wherein the same set of S-measurements is used for calibration and imaging of the contents.

12. The method of claim 10, wherein the background model comprises content height at a wall of the container, cone angle formed by the contents, and bulk average permittivity of the contents.

13. The method of claim 12, wherein the container comprises a grain storage bin, and the contents is grain.

14. The method of claim 10, wherein the calibration coefficients correspond to channel loss and phase shift.

15. The method of claim 10, wherein determining calibration coefficients comprises performing a minimization with the measurement data and the background model.

16. The method of claim 15, wherein the minimization comprises an L2 norm minimization.

17. The method of claim 10, further comprising performing full inversion based on the calibrated scattered field measurements and the measurement data and one or more of providing an image map of the contents or activating a moisture-affecting device based on the full inversion.

18. The method of claim 17, wherein the image map comprises a permittivity map.

19. A non-transitory, computer readable medium comprising instructions, that when executed by one or more processors, causes the one or more processors to:
receive measurement data of a container with contents stored within the container;
perform a phaseless parametric inversion on the measurement data to provide a background model;
determine calibration coefficients for each of a plurality of channels based on the measurement data and the background model; and
determine calibrated scattered field measurements based on the background model and the calibration coefficients.

20. The non-transitory, computer readable medium of claim 19, further comprising instructions, that when executed by the one or more processors, causes the one or more processors to perform full inversion based on the calibrated scattered field measurements and the measurement data and one or more of provide an image map of the contents or activate a moisture-affecting device based on the full inversion.
